(19) United States
(12) Patent Application Publication
(10) Pub. No.: US 2018/0221177 A1
(43) Pub. Date: Aug. 9, 2018

(54) DIGITAL INTERFACE SYSTEM AND METHOD

(71) Applicants: David Kaltenbach, Berlin (DE); Max Mahal, Berlin (DE); Lucas Rex, Berlin (DE)

(72) Inventors: David Kaltenbach, Berlin (DE); Max Mahal, Berlin (DE); Lucas Rex, Berlin (DE)

(21) Appl. No.: 15/424,268

(22) Filed: Feb. 3, 2017

(51) Int. Cl. A61F 2/72; A61B 5/0488; A61B 5/00; A61F 2/58; G06F 3/038; G06F 3/01; G06F 3/0346; G06F 1/16

(52) U.S. Cl. CPC A61F 2/72; A61B 5/04888; A61B 5/6824; A61F 2/586; A61F 2002/704; G06F 3/017; G06F 3/0346; G06F 1/163; G06F 3/0383

(57) ABSTRACT

A digital interface systems for controlling a computing device and prosthesis technology for compensating the loss of a limb. In one example, a digital interface system for translating a myoelectrical signal into a control command for a computing device is provided. The system includes a myoelectrical sensor arrangement, which is configured for sensing at least one myoelectrical signal of a muscle and/or of a nerve of a muscle. Additionally, the system has a pointing device adapted for detecting a two-dimensional motion of its own wherein the pointing device has at least one optical location-sensor. Additionally, the sensed myoelectrical signal of the myoelectrical sensor arrangement and the pointing signal of the pointing device can be fed into a controlling means.

### BACKGROUND OF THE INVENTION

The subject matter described herein relates generally to digital interface systems for controlling a computing device, and more particularly, to prosthesis technology for compensating the loss of a limb, for example a forearm, of a person.

Generally, for controlling a computing device—for example a personal computer—a computer mouse is used by a large part of computer users. When using a computer mouse, said computer mouse is usually moved on a planar surface, wherein said movement is used to direct a computer cursor on a user surface of said computer, for example on a desktop of the computer.

In the event of a loss of a hand a person is not any longer capable of controlling a computer mouse with the lost hand. In particular, a computer mouse needs to be handled with the remaining hand, which negatively effects the overall productivity and comfort when working with a computer.

### BRIEF DESCRIPTION OF THE INVENTION

In one aspect, a digital interface system for translating a myoelectrical signal into a control command for a computing device provided. Said myoelectrical signal originates from a muscle or a nerve of a body of a person or an animal. The system comprises a myoelectrical sensor arrangement, which is configured for sensing at least one myoelectrical signal of a muscle and/or of a nerve of a muscle. Additionally, the system has a pointing device adapted for detecting a two-dimensional motion of its own relatively to a support surface when being placed and moved on the support surface and in parallel to the support surface, wherein the pointing device has at least one optical location-sensor. Additionally, controlling means are provided by the system, wherein the sensed myoelectrical signal of the myoelectrical sensor arrangement and the pointing signal of the pointing device can be fed into the controlling means. For such purpose, the myoelectrical sensor arrangement, the pointing device and the controlling means are configured such that the myoelectrical sensor arrangement and the pointing device are connectable to the controlling means for signal transfer.

According to a non-limiting embodiment of the aspect mentioned above, the pointing device and the myoelectrical sensor arrangement are configured such that they can be attached to a forearm, upper arm and/or shoulder portion of a person, in particular to a remaining residual limb of a forearm, a of an upper arm or of a shoulder portion of an amputated person. Preferably, the myoelectrical sensor arrangement is placed on an area of the residual limb, which still contains mentally controllable muscles and/or nerves.

According to one embodiment, the controlling means interpret the signal from the myoelectrical sensor arrangement and the signal from the pointing device, wherein at least one signal from the myoelectrical sensor arrangement is mapped to a control command for the computing device. For example, said control command can be for example a left-click′ or ‘right-click’ of a computer mouse, ‘scrolling-up’ and ‘scrolling-down’ in a computer window, etc. For such purpose the controlling means are configured to be able to understand and work with the transmitted signals, in particular with the sensed myoelectrical signal provided by the myoelectrical sensor arrangement and with the pointing signal provided by the pointing device. In particular, the controlling means combine the signal of the pointing device with the signal of the myoelectrical sensor arrangement, it maps the signal of the myoelectrical sensor to a specific control command for the computing device, and provides this combined signal as a control data stream to the computing device. In other words, the control device performs a fusion of the myoelectrical signal and the pointing signal by sensor-fusion. As an example, such signal/sensor-fusion is performed when executing a ‘drag-and-drop’-procedure.

According to a non-limiting embodiment, the computing device itself comprises the controlling means by providing adequate controlling software. Hence, it is disclosed, that the controlling means can be embodied as software which runs on the very computing device and shall be controlled by the digital interface system. Thus, the software forms in combination with the computing device itself a control device for interpreting, handling and processing the sensed myoelectrical signal and the pointing device signal.

Additionally and/or alternatively, the digital interface system explicitly comprises a separate controlling device, wherein the myoelectrical sensor arrangement and the pointing device are connected to the control device for signal transfer. In particular, the sensed myoelectrical signal is transferred from the myoelectrical sensor arrangement to the control device, and the pointing signal is transmitted from the pointing device to the control device.

According to a particular embodiment, the control device is either mounted to the pointing device or to the myoelectrical sensor arrangement. Therefore, the digital interface system comes with at least, in particular not more than, two separate components, wherein the myoelectrical sensor arrangement, optionally integrated with the control device, forms one component, which needs to be attached to an area of the body, where a myoelectrical signal can be detected by a myoelectrical sensor. The pointing device forms the second component, optionally jointly embodied with the control device.

In a said configuration—but not limited to it—the pointing device and the myoelectrical sensor arrangement can be connected to the computing device for transmitting the control data stream either via cable or wirelessly.

In particular, the myoelectrical sensor can be formed as a bracelet, which—for example—is configured to be attached to a forearm or a residual limb of a forearm of a person. Such configuration comes with major benefits in means of comfort and usability.

According to a further non-limiting embodiment, the pointing device is embodied in form of a wristband. Such configuration comes with the advantage, that the pointing device can be controlled accurately.

Further aspects, advantages and features of the first aspect of the present invention are apparent from the dependent claims, the description and the accompanying drawings.

According to a second aspect, a prosthesis for translating at least one myoelectrical signal of a muscle and/or of a nerve into a control command for a computing device is provided. Such prosthesis comprises a prosthesis body, a myoelectrical sensor arrangement, a pointing device and a control device. The myoelectrical sensor arrangement is configured for sensing at least one myoelectrical signal of a muscle and/or of a nerve. The pointing device is adapted for detecting a two-dimensional motion of the pointing device itself relatively to a support surface. For providing one single component, the myoelectrical sensor arrangement and the pointing device are connected to the control device, wherein at least the myoelectrical sensor arrangement and at least the pointing device are integrally formed in/on the prosthesis body. By this it can be achieved, that an amputated person may obtain an extended functionality of the prosthesis. In particular, the person is able to control a computing device by using said prosthesis, wherein a cursor of the computing device is controlled via the signal of the pointing device and specific control commands—expressed via the cursor and/or at the location of the cursor —, for example “left click”, “right-click” and/or “zoom in/out”, etc., are executed in alignment with the sensed myoelectrical signal and the pointing signal.

According to an embodiment of the second aspect, the control device is configured for mapping at least one sensed myoelectrical signal to at least one control command for the computing device. In particular, different myoelectrical signals measured from a plurality of muscles and/or nerves on/in a residual limb of the person are mapped to different specific control commands for the computing device.

In order to provide an effective control over the computing device, the control device performs sensor-fusion with regard to the signals of the pointing device and of the myoelectrical sensor arrangement.

According to an exemplary embodiment, the prosthesis body is provided for replacing an amputated limb of a person, for example a hand, partially a forearm and a hand or partially the entire arm of a person. For such reason, the prosthesis body is adapted for being attached to a residual limb portion of the person.

Optionally, the prosthesis can have an artificial hand with artificially movable fingers, wherein a drive of at least one of the fingers is controlled by using the sensed myoelectrical signal. That means, if the user of the prosthesis does not want to use/control a computer but needs the artificial hand for grabbing purposes, the drives of the artificially movable fingers are activated or deactivated in accordance to a myoelectrical signal of a muscle and/or of a nerve.

According to a non-limiting development of the optional embodiment as mentioned above, the control device is configured such that the prosthesis can be operated in a first operational mode and in a second operational mode. During the first operational mode a drive of at least one artificially movable finger is activated or deactivated based on a myoelectrical signal, wherein during the second operational mode the myoelectrical signal is used for providing at least one control command for the computing device. In particular, during second operational mode the drives of the artificially movable fingers are completely cut off and therefore deactivated and do not perform any movement based on the myoelectrical signal.

Further aspects, advantages and features of the second aspect of the present invention are apparent from the dependent claims, the description and the accompanying drawings.

In yet a third aspect, a method for operating a digital interface system as presented in the first aspect and/or for operating a prosthesis of the second aspect of the invention is presented, wherein the system and/or the prosthesis comprises a myoelectrical sensor arrangement, a pointing device and controlling means or a control device for translating a myoelectric signal of a muscle and/or of a nerve into a control command for a computing device. Said method comprises the following steps:
a) measuring at least one myoelectrical signal of a muscle and/or of a nerve;
b) detecting a two-dimensional motion of the pointing device being placed on a support surface relatively to the support surface by using at least one optical location-sensor;
c) mapping at least one myoelectrical signal with at least one control command for the computing device; and
d) translating the detected two-dimensional motion into at least one curser movement for a cursor of the computing device.

By executing the steps a) to d) of the method mentioned above, a computing device like a personal computer can be controlled such that the cursor of the computing device is moved according to the two-dimensional motion of the pointing device, wherein at least one specific control command, for example a left-click′ or a ‘right-click’ can be executed at a specific cursor position on the computing device. In particular, the method as mentioned does not require and/or include the usage of a computer mouse.

In the course of an additional step e) the myoelectrical signal and the signal of the two-dimensional motion (pointing signal) are combined with each other and sent via cable or wirelessly in form of a control data stream to the computing device.

Specifically—but not limited to—the use and/or presence of a computer mouse can be explicitly excluded from system according to the first aspect and from the execution of the mentioned method according to the third aspect of the invention.

Further aspects, advantages and features of the second aspect of the present invention are apparent from the dependent claims, the description and the accompanying drawings.

### BRIEF DESCRIPTION OF THE DRAWINGS

A full and enabling disclosure including the best mode thereof, to one of ordinary skill in the art, is set forth more particularly in the remainder of the specification, including reference to the accompanying figures wherein:

FIG. 1 is a schematic display of an embodiment of the digital interface system according to the first aspect of the invention.

FIG. 2 is a perspective view on an embodiment of the wristband according to FIG. 1.

FIG. 3 is a schematic display of an embodiment of a prosthesis according to the second aspect of the invention.

FIG. 4 represents a simplified mapping scheme for mapping a specific myoelectrical signal to a specific control command.

### DETAILED DESCRIPTION OF THE INVENTION

Reference will now be made in detail to the various embodiments, one or more examples of which are illustrated in each figure. Each example is provided by way of explanation and is not meant as a limitation. For example, features illustrated or described as part of one embodiment can be used on or in conjunction with other embodiments to yield yet further embodiments. It is intended, that the present disclosure includes such modifications and variations.

One embodiment described herein includes a digital interface system for translating at least one myoelectrical signal of a muscle and/or of a nerve into a control command for a computing device. Said system includes a myoelectrical sensor arrangement, a pointing device and controlling means. The myoelectrical sensor arrangement and the pointing device can be connected to the controlling means for transferring a measured myoelectrical signal and a pointing signal of the pointing device to the controlling means. The system facilitates to control a cursor of a computing device with the help of the pointing device, and additionally to provide a specific control command to the computing device via the myoelectrical sensor arrangement and the controlling means. For example, an amputated person having a forearm stump without a hand would use said digital interface system such, that he/she applies the myoelectrical sensor arrangement to the stump of the forearm and attaches the pointing device directly on the stump or on a prosthesis the person is wearing, preferably at a wrist area of the prosthesis. With the help of the pointing device the user is able to control a cursor of the computing device. In case there were not any additional measures, the person would not be able to execute any control commands like ‘left click’, ‘right-click’, ‘scrolling up’ and/or ‘scrolling down’. However, with the help of the described embodiment of the digital interface system the execution of such commands can be triggered by a myoelectric signal measured on/in a remaining muscle in the residual limb of the person, wherein the measurement is performed with the myoelectric sensor arrangement. For such purpose, the controlling means perform a sensor fusion of the signal of the myoelectrical sensor arrangement and the signal of the pointing device; that means, the myoelectrical signal and the pointing signal are aligned with each other and combined into one control data stream for controlling the computing device. With the help of the described system an arm-/hand-amputated person is enabled—for the first time—to control a computing device naturally and without major restrictions in a similar way as a computing device can be controlled with a computer mouse.

Another embodiment, provided in the following, includes a prosthesis comprising a myoelectrical sensor arrangement, a pointing device and a control device—preferably in an integrated manner. Analogously to the embodiment described above, the invention provides the ability to easily and comfortably control a computing device to a person who suffered an amputation. In particular, the technical means for achieving the effect of the invention are discreetly integrated into the prosthesis body of the prosthesis. Hence, the usage of the described embodiment is esthetically appealing, simple and efficient.

As used herein, the term “myoelectrical signal of a muscle and/or of a nerve” is intended to be representative of any measurable electric voltage being present in an activated muscle, in an activated nerve and/or in an activated muscle-nerve-transition area. Such measured electrical signal can be obtained by myoelectrical and/or neuro-electrical measuring methods.

The term “myoelectrical signal” may refer to both where applicable, firstly to the measured electric voltage within the muscle/nerve, secondly to the signal provided by the myoelectrical sensor arrangement representing the measured signal.

As used herein, the term “myoelectrical sensor arrangement” is intended to be representative of any arrangement comprising at least one myoelectrical or neuro-electrical sensor. Said sensor is configured and suitable to measure an electric voltage being present in a muscle, in a nerve and/or in a muscle-nerve-transition area. According to one embodiment the myoelectrical sensor of the myoelectrical sensor arrangement is adapted for being placed on an outer surface of a limb or residual limb of a person. According to an additional or alternative embodiment the myoelectrical sensor is placed within a limb or residual limb of a person.

As used herein, the term “pointing device is adapted for detecting an own two-dimensional motion relatively to a support surface when being placed and moved on and in parallel to the support surface” is to be understood, that it is crucial for the desired functionality of the pointing device to be placed on a surface. When the pointing device is moved in parallel on said surface the so-called pointing signal is caused by the relative movement of the pointing device with regards to the surface.

In particular, but not limited to, the pointing device is configured to exclusively detect a mainly two-dimensional translation movement being performed relatively to the surface on which the pointing device is placed on. Thus, the pointing device is not capable of additionally detecting a three-dimensional motion.

Additionally, according to a non-limiting embodiment, the pointing device and/or the digital interface system and/or the prosthesis do not include a gyroscopic sensor for detecting a change in the position.

As used herein, the term “computing device” includes all technical devices having at least one computing core, and which are required to communicate with humans, in particular in means of obtaining commands. Thus, the term “computing device” includes, but not limited to it, personal computers, notebooks, smart phones, phones, car computers, machine controls, etc.

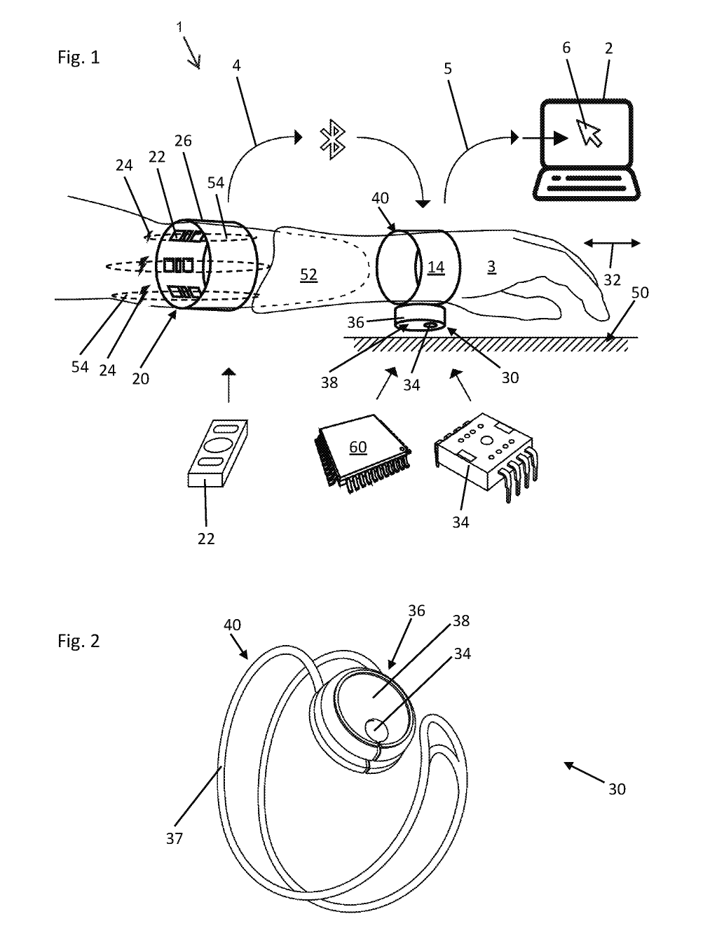

FIG. 1 is a schematic view on an embodiment of the digital interface system 1. According to the present embodiment, the digital interface system 1 is used in context with a passive prosthesis 3, which is attached to a residual limb 52. A passive prosthesis 3 does not provide any advanced functionality in means of providing an artificial ability to move, but its main purposes are to provide an optical dummy for the lost limb in a social context, to act as a balance for the body weight distribution of the person and for executing further raw physiological tasks. The digital interface system 1 is a suitable embodiment specialized for and used together with a passive prosthesis 3. Mainly, the digital interface system 1 includes a wristband 40 and a bracelet 26, wherein the wristband 40 comprises a pointing device 30, and wherein the bracelet 26 includes the myoelectrical sensor arrangement 20.

The wristband 40—also shown in greater detail in FIG. 2—comprises an attachment band 37 and a support portion 36, wherein the support portion 36 can be attached to the prosthesis 3 with the help of the attachment band 37, which for example is made from an elastic material. The support portion 36 is designed for supporting the weight of the prosthesis 3 and/or of the residual limb 42 on a support surface 50, for example a table, for housing at least an optical location-sensor 34 and for ensuring a proper functioning of said optical location sensor 34. According to the present embodiment, the support portion 36 including the optical sensor 34 can be functionally understood as pointing device 30. The shown embodiment discloses, that a control device 60 is also arranged in the support portion 36.

Alternatively, the control device 60 could be arranged to the bracelet 26, too.

The bracelet 26 including the myoelectric sensor arrangement 20 is attached to a portion of the residual limb 42, which contains actively controllable muscles 54 generating measurable myoelectric signals 24. In order to obtain a proper measurement of a myoelectric signal 24, the bracelet 26 must be placed on the residual limb 52 such, that a myoelectrical sensor 22 is suitably placed on the targeted remaining muscle 54. The myoelectric sensor arrangement 20 according to FIG. 1 comprises a plurality of myoelectric sensors 20, which send a myoelectric signal 24 from a plurality of remaining muscles 54. If the user is conducting specific phantom hand gestures, the myoelectric sensor arrangement 20 senses different types of specific myoelectric signals 24.

The pointing device 30, the myoelectric sensor arrangement 20 and the control device 60 are equipped with suitable wireless connection means, for example Bluetooth, such that the myoelectric sensor arrangement 20 can communicate with the control device 60 for signal transfer 4. Additionally, the pointing device 30 is connected to the control device 60 for signal transfer 4, wherein the control device 60 comprises suitable communication means in order to establish a connection for transmitting the control data stream 5 to the computing device 2. Both components, the wristband 40 having the pointing device 30 and the control device 60 and the bracelet 26 including the myoelectric sensor arrangement 20, have suitable energy storage devices.

An exemplary, non-limiting usage of the digital interface system 1 can be described as follows: before starting to work with a computing device 2, for example, a personal computer, the user wearing a passive prosthesis 3 places the bracelet 26 on the residual limb portion 52. The residual limb 52 is represented in FIG. 1 by a dotted line. Furthermore, the wristband 40 having the pointing device 20 is attached to the wrist of the passive prosthesis 3. When being switched on, the myoelectric sensor arrangement 20 connects wirelessly to the control device 60, and the pointing device 30 establishes a connection to the control device 60. Furthermore, a data connection for transmitting the control data stream 5 to the computing device 2 is established between the control device 60 and the computing device 2.

When moving the wristband 40 over the support surface 50 in the course of a two-dimensional motion 32, in particular by shoving the planar surface 38 in parallel over the support surface 50, a relative movement of the pointing device 40 with regard to the support surface 50 is detected by the optical location-sensor 34. The control device 60 translates said relative motion 32 into a movement of a cursor 6 on a screen of the computing device 2.

In order to provide an additional control command 62, or example a left-click′ or ‘right-click’ of a computer mouse, ‘scrolling-up’ and ‘scrolling-down’ in a computer window, etc., different myoelectric signals 24a, 24b, 24c, 24d, 24e and 24f can be detected and mapped to related control commands 62. The different myoelectric signals 24 origin from different phantom gestures executed by the user, wherein different muscles 54 in the residual limb 52 are activated. Hence it is possible to create a specific myoelectric signal 24 by performing a specific phantom gesture, wherein each identified specific myoelectric signal 24 is assigned to a specific control command 62 of the computing device 2. Thus, specific control command 62 can be initiated on the computing device 2 by executing a specific related phantom gesture.

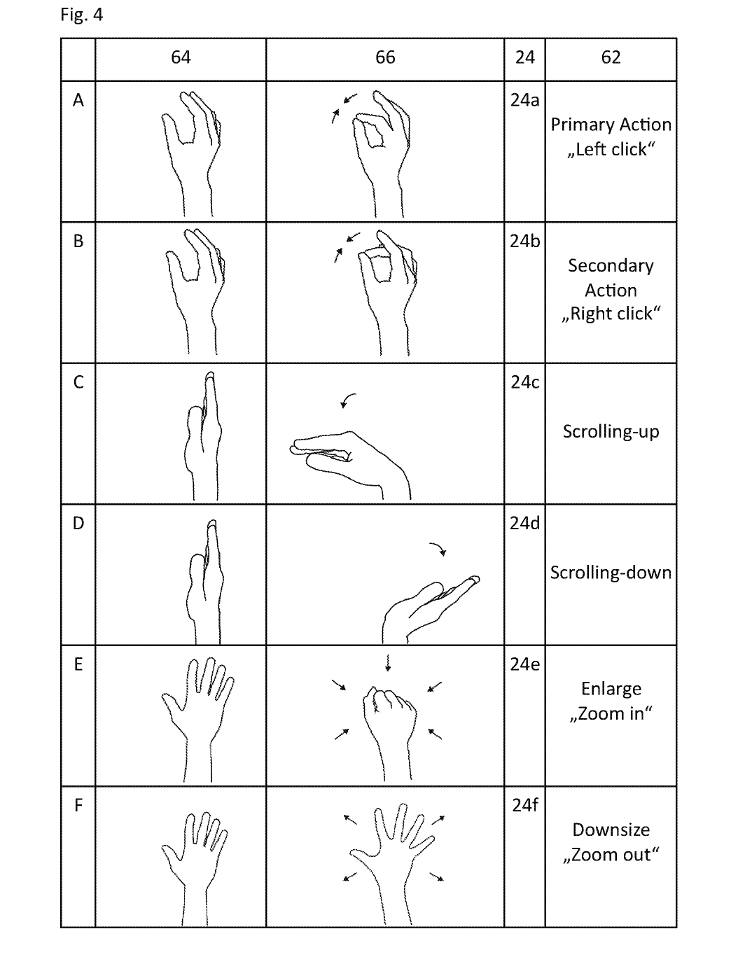

FIG. 4 shows a possible embodiment for a mapping structure for assigning specific control commands 62 to specific gestures.
Row A: “bringing thumb and index finger in contact with each other” 66a, wherein the movement is referenced to relaxed initial hand position 64; gesture 66a is assigned to a control command 62a ‘left click’;
Row B: “bringing thumb and middle finger in contact with each other” 66b, wherein the movement is referenced to a relaxed initial hand position 64b; gesture 66b is assigned to a control command 62b ‘right click’;
Row C: “abduction of the hand to the left side” or “left-waving” 66c, wherein the movement is referenced to a straight initial hand position 64c; gesture 66c is assigned to a control command 62c ‘scrolling up’;
Row D: “abduction of the hand to the right side” or “right” 66d, wherein the movement is referenced to a straight initial hand position 64d; gesture 66d is assigned to a control command 62d ‘scrolling down’;
Row E: “making a fist” 66e, wherein the movement is referenced to a relaxed open hand palm position 64e; gesture 66e is assigned to a control command 62e ‘zoom-in’;
Row F: “spreading the fingers wide open” 66f, wherein the movement is referenced to a relaxed open hand palm position 64f; gesture 66f is assigned to a control command 62f ‘zoom-out’.

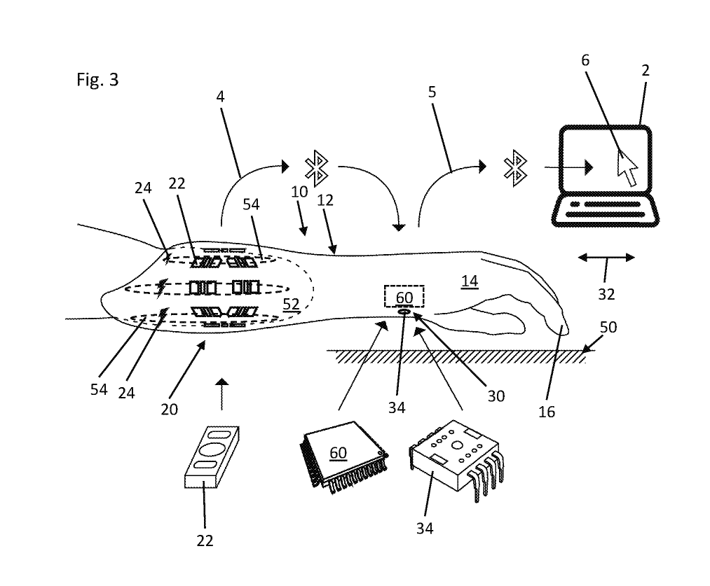

FIG. 3 introduces an embodiment of a prosthesis 10 according to the second aspect of the invention, wherein the digital interface system 1 similar to the first aspect of the invention is integrated in a prosthesis body 12 of the prosthesis 10. The myoelectrical sensor arrangement 20 and the pointing device 30 are fully located in the prosthesis body 12, so that preferably both are not visible as such.

According to FIG. 3 the residual limb 52 is a stump of a forearm. Thus, the prosthesis 10 would be formed as a whole forearm. In such residual limb 52 some muscles 54 may remain active and mentally controllable. Myoelectrical signals 24 generated by these muscles 54 can be sensed by the myoelectrical sensor 22 of the myoelectrical sensor arrangement 20. For that reason, the myoelectrical sensor arrangement 20 is located at a portion of the prosthesis 10, which is used to mount the prosthesis 10 to the residual limb 52. Hence, the prosthesis 10 comprises a portion, which is designed to receive—at least partially—the residual limb 52. Usually, a prosthesis 10 has an inner volume for such purpose, which is fitted to the specific form of the residual limb 52.

Additionally, the prosthesis body 12 houses the pointing device 30 and the control device 60. Preferably, both components are arranged in the prosthesis body 12 such, that they are not exceedingly visible from the outside. However, the pointing device 30 may require a minor opening, preferably on the lower side of the wrist of the prosthesis body 12, in order to be enabled to detect a relative motion 32 with regard to the support surface 50. This applies particularly, if the pointing device 30 comprises an optical sensor 34, wherein the optical sensor 34 would be arranged in the near surrounding of the opening in the artificial skin of the prosthesis 10.

The control device 60 can be arranged completely hidden within the prosthesis body 12, wherein the control device is connected to the myoelectric sensor arrangement 20 and with the pointing device 34 for signal transfer 4 via cable or wirelessly, for example by Bluetooth.

For transmitting the control data stream 5—comprising the mapped control commands 62 and the intended cursor movements provided by the pointing device 30—the control device 60 is preferably equipped with wireless communication means for establishing a suitable connection with the computing device 2.

FIG. 3 includes an additional embodiment, wherein movable fingers 16 of the artificial hand 14 of the prosthesis 10 are equipped with drives in order to execute movements. For this purpose the control device 60 can apply a first operational mode and a second operational mode. In the first operational mode the myoelectrical signal 24—sensed by the myoelectrical sensor arrangement 20—is used for activating or deactivating a drive of the artificial fingers 16. During the second operational mode the myoelectrical signals 24 are mapped to control commands 62 as described above.

According to a preferred elaboration of the embodiment as mentioned above, the control means 60 include additional detecting means for recognizing, if the prosthesis 10—in the very moment—is intended to be used for controlling the computing device 2, or if it is intended to be used for grabbing something. For example, such detection means may be connected to the control device 60, if the prosthesis 10 is in a suitable position on a support surface 50 for controlling the computing device 2, or if the prosthesis 10 is moving around for example if the related person is walking. In order to detect the position of the prosthesis 10 the optical sensor 34 could be used.

The embodiment shown in FIG. 3 is adapted for being placed on a residual limb 52 of a forearm. However, the person skilled in the art is aware of the possibility to modify the design principle of the shown embodiments for other residual limb configurations. For example, in case the entire forearm is lost and only a stump of the upper arm remains as residual limb, the prosthesis 10 would be designed accordingly, wherein the myoelectric sensor arrangement 20 would be suitably placed for detecting a myoelectric signal of the biceps and/or triceps, etc.

Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. While various specific embodiments have been disclosed in the foregoing, those skilled in the art will recognize that the spirit and scope of the claims allows for equally effective modifications. Especially, mutually non-exclusive features of the embodiments described above may be combined with each other. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A digital interface system for translating at least one myoelectrical signal of a muscle and/or of a nerve into a control command for a computing device comprising:
a myoelectrical sensor arrangement configured for sensing at least one myoelectrical signal of a muscle and/or of a nerve;
a pointing device adapted for detecting an own two-dimensional motion relatively to a support surface when being placed and moved on and in parallel to the support surface, wherein the pointing device has at least one optical location-sensor; and
controlling means, wherein the myoelectrical sensor arrangement, the pointing device and the controlling means are configured such that the myoelectrical sensor arrangement and the pointing device are connectable to the controlling means for signal transfer.

2. The digital interface system according to claim 1, wherein the controlling means are configured for mapping at least one myoelectrical signal with at least one control command for the computing device.

3. The digital interface system according to claim 1, wherein the controlling means are embodied as a control device, which is mounted to the pointing device (30) or to the myoelectrical sensor arrangement, and wherein the myoelectrical sensor arrangement and the pointing device are connected to the control device (60) for signal transfer.

4. The digital interface system according to claim 1, wherein the pointing device has a support portion with a planar surface, wherein the planar surface is adapted to be placed in parallel on the support surface for operating the pointing device.

5. The digital interface system according to claim 1, comprising a wristband, wherein the pointing device is installed in the wristband.

6. The digital interface system according to claim 5, comprising wireless connection means for connecting the pointing device with the controlling means for signal transfer.

7. The digital interface system according to claim 1, comprising a bracelet, wherein the myoelectrical sensor arrangement is installed in the bracelet.

8. The digital interface system according to claim 7, comprising wireless connection means for connecting the myoelectrical sensor arrangement with the controlling means for signal transfer.

9. A prosthesis for translating at least one myoelectrical signal of a muscle and/or of a nerve into a control command for a computing device comprising:
a prosthesis body for replacing an amputated limb of a person adapted for being attached to a residual limb portion of the person;
a myoelectrical sensor arrangement configured for sensing at least one myoelectrical signal of a muscle and/or of a nerve;
a pointing device adapted for detecting an own two-dimensional motion relatively to a support surface when being placed and moved on and in parallel to the support surface; and
a control device, wherein the myoelectrical sensor arrangement and the pointing device are connected to the control device for signal transfer, and wherein the control device, the myoelectrical sensor arrangement and the pointing device are integrated with the prosthesis body.

10. The prosthesis according to claim 9, wherein the control device is configured for mapping at least one myoelectrical signal with at least one control command for the computing device.

11. The prosthesis according to claim 9, wherein the pointing device has at least one optical location-sensor.

12. The prosthesis according to claim 9, wherein the prosthesis is adapted for at least partially replacing a hand, a forearm and/or an upper arm of a person, and wherein the residual limb is a stump of a forearm, of an upper arm and/or of a shoulder of a person.

13. The prosthesis according to claim 12, comprising an artificial hand with artificially movable fingers, wherein a drive of at least one of the fingers is controlled by using the myoelectrical signal of the muscle and/or of the nerve.

14. The prosthesis according to claim 13, wherein the control device is configured to operate the prosthesis in a first operational mode and in a second operational mode, wherein in the first operational mode a myoelectrical signal activates the drive of the finger, and wherein in the second operational mode the myoelectrical signal is translated into the control command for the computing device.

15. The prosthesis according to claim 9, comprising wireless connection means for connecting the control device with the computing device for signal transfer.

16. A method for operating a digital interface system having a myoelectrical sensor arrangement, a pointing device and controlling means for translating a myoelectrical signal of a muscle and/or of a nerve into a control command for a computing device, comprising the steps:
a) measuring at least one myoelectrical signal of a muscle and/or of a nerve;
b) detecting a two-dimensional motion of the pointing device being placed on a support surface relatively to the support surface by using at least one optical location-sensor;
c) mapping at least one measured myoelectrical signal with at least one control command for the computing device; and
d) translating the detected two-dimensional motion into at least one curser movement for a cursor of the computing device.

17. The method according to claim 16, wherein the digital interface system is embodied in a prosthesis for a forearm and an artificial hand including automatically movable fingers, wherein the controlling means can operate the digital interface system in a first mode and in a second mode, wherein in the first mode a myoelectrical signal is used for activating a drive of the finger, and wherein in the second mode the myoelectrical signal is translated into the control command for the computing device.

18. The method according to claim 16, comprising the step: detecting an operating status and/or a position of the pointing device, wherein the digital interface system is operated in the first mode when the pointing device is non-operational or lifted from the support surface, and wherein the digital interface system is operated in the second mode when the pointing device is operational and/or placed on the support surface.

19. A system comprising:
a myoelectrical sensor arrangement that senses at least one myoelectrical signal of a muscle or a nerve representative of a control command for a computing device; and
a pointing device that detects two-dimensional motion and provides a pointing device output signal representative of the two-dimensional motion.

20. The system of claim 19, comprising:
a bracelet that couples the myoelectrical sensor arrangement to a limb; and
a wristband that couples the pointing device to a prothesis.

21. The system of claim 19, comprising:
a control device that provides an output control signal based on the myoelectrical sensor arrangement output signal and/or the pointing device output signal.
